(19) United States
(12) Patent Application Publication
(10) Pub. No.: US 2025/0352459 A1
(43) Pub. Date: Nov. 20, 2025

(54) TOPICAL COSMETIC BALM FORMULATION

(71) Applicant: Tyson Salomon, Los Angeles, CA (US)

(72) Inventor: Tyson Salomon, Los Angeles, CA (US)

(21) Appl. No.: 18/668,186

(22) Filed: May 19, 2024

(51) Int. Cl. A61K 8/9789; A61Q 5/12; A61Q 19/00; A61Q 19/08; G06T 19/00

(52) U.S. Cl. CPC A61K 8/9789; A61Q 5/12; A61Q 19/007; A61Q 19/08; G06T 19/006; A61K 2800/5922; A61K 2800/805

(57) ABSTRACT

The embodiments disclose a body balm including a composition of ingredients comprising organic shea butter in a predetermined range of concentration, organic jojoba oil in a predetermined range of concentration, organic vitamin E oil in a predetermined range of concentration, organic calendula oil in a predetermined range of concentration, organic sweet almond oil in a predetermined range of concentration, the organic lavender essential oil in a predetermined range of concentration, organic Palo Santo essential oil in a predetermined range of concentration and wherein the composition of ingredients is made up into of all organic vegan balm product types including a body balm, a body glaze, and body butter.

### BACKGROUND

Many current moisturizers contain synthetic ingredients that can have negative results for sensitive skin or allergies to any of the ingredients. Users of moisturizers are sometimes unaware of the synthetic ingredients and whether they have allergies or irritations to those ingredients.

### BRIEF DESCRIPTION OF THE DRAWINGS

FIG. 1 shows a block diagram of an overview of an all-organic vegan body balm of one embodiment.

FIG. 2 shows a block diagram of an overview of processing the all-organic vegan body balm ingredients of one embodiment.

FIG. 3 shows a block diagram of an overview of the ingredient characteristics of one embodiment.

FIG. 4 shows for illustrative purposes only an example of combining organic ingredients of one embodiment.

FIG. 5 shows a block diagram of an overview of ingredients by weight of one embodiment.

FIG. 6 shows for illustrative purposes only an example of a user sharing application results on social media of one embodiment.

FIG. 7 shows a block diagram of an overview of the all-organic vegan balm product types of one embodiment.

### SUMMARY OF THE INVENTION

The embodiments of the present invention disclose a body balm for use as a whole-body hydrating and softening application. In one embodiment, the body balm includes a composition of ingredients where the composition of all the ingredients are organic and vegan balm product types. The ingredients of the body balm can include a body glaze, and a skin hydrator and softener, comprising organic shea butter in a predetermined range of concentration, organic jojoba oil in a predetermined range of concentration, organic vitamin E oil in a predetermined range of concentration, organic calendula oil in a predetermined range of concentration, organic sweet almond oil in a predetermined range of concentration, organic lavender essential oil in a predetermined range of concentration, organic Palo Santo essential oil in a predetermined range of concentration.

### DETAILED DESCRIPTION OF THE INVENTION

In the following description, reference is made to the accompanying drawings, which form a part hereof, and which are shown by way of illustration a specific example in which the invention may be practiced. It is to be understood that other embodiments may be utilized, and structural changes may be made without departing from the scope of the present invention.

### General Overview:

It should be noted that the descriptions that follow, for example, the topical cosmetic balm formulation and body balm skin hydrator and softener formulation are described for illustrative purposes, and the underlying composition, formulation, methods, and systems can apply to any number and multiple types of topical cosmetic balm formulation and body balm hydrating and softening ingredients and equivalent moisturizing dry skin treatments. In one embodiment of the present invention, the topical cosmetic balm formulation is a body balm skin hydrating and softening formulation that includes various methods and devices that can be configured using multiple ingredients. The topical cosmetic balm formulation can be configured to be clear or partially translucent and can include organic ingredients and can also be configured to include vegan ingredients using the present invention.

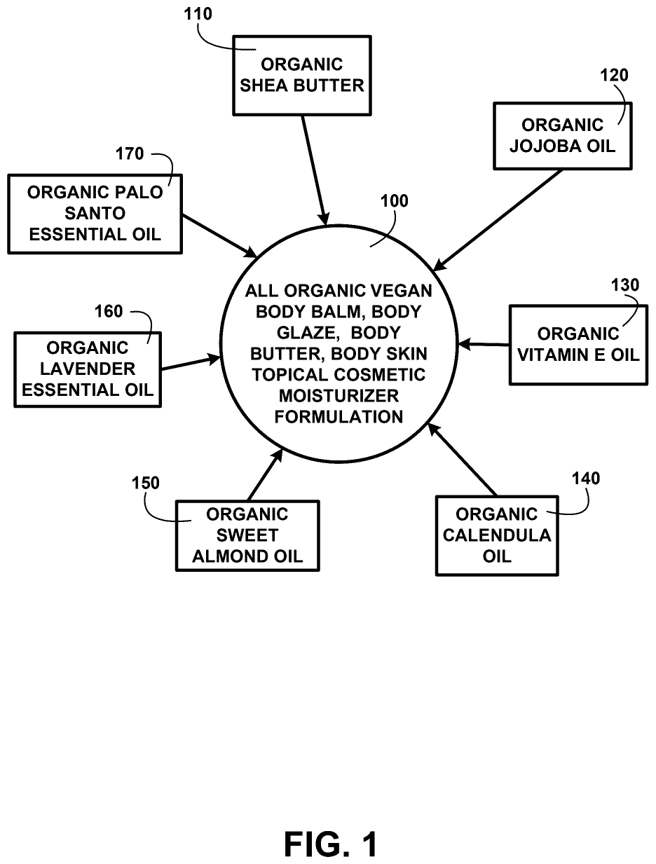

FIG. 1 shows a block diagram of an overview of an all-organic vegan body balm of one embodiment. FIG. 1 shows an all-organic vegan body balm, body glaze, body butter, and body skin topical cosmetic balm formulation 100. The all-organic vegan body balm includes organic natural non-animal ingredients. The organic natural non-animal ingredients include organic shea butter 110, organic jojoba oil 120, organic vitamin E oil 130, organic calendula oil 140, organic sweet almond oil 150, organic lavender essential oil 160, and organic Palo Santo essential oil 170. The all-organic vegan body balm ingredients have characteristics that aid in hydrating and softening the skin of one embodiment.

### DETAILED DESCRIPTION

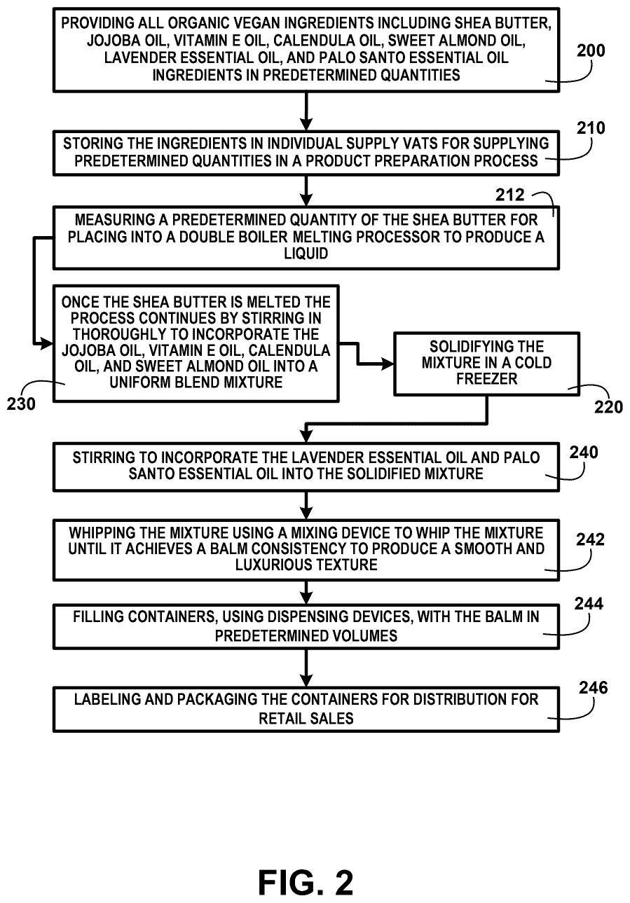

FIG. 2 shows a block diagram of an overview of processing the all-organic vegan body balm ingredients of one embodiment. FIG. 2 shows providing all-organic vegan ingredients including shea butter, jojoba oil, vitamin E oil, calendula oil, sweet almond oil, lavender essential oil, and Palo Santo essential oil ingredients in predetermined quantities 200. The process begins with storing the ingredients in individual supply vats for supplying predetermined quantities in a product preparation process 210. The product preparation process begins with measuring a predetermined quantity of the shea butter for placing into a double boiler melting processor to produce a liquid 212. Once the shea butter is melted the process continues by stirring in thoroughly to incorporate the jojoba oil, vitamin E oil, calendula oil, and sweet almond oil into a uniform blend mixture 230.

The uniform blend mixture includes solidifying the mixture in a device that allows the mixture to cool considerably, such as a freezer 220. The processing continues by stirring to incorporate the lavender essential oil and Palo Santo essential oil into the solidified mixture 240. All of the ingredients when mixed undergo whipping the mixture using a mixing device to whip the mixture until it achieves a balm consistency to produce a smooth and luxurious texture 242. The whipped balm is pumped from a mixing vat for filling into containers, using dispensing devices, with the balm in predetermined volumes 244. Once filled the filled containers are sealed and proceed for labeling and packaging the containers for distribution for retail sales 246 of one embodiment.

### Ingredient Characteristics:

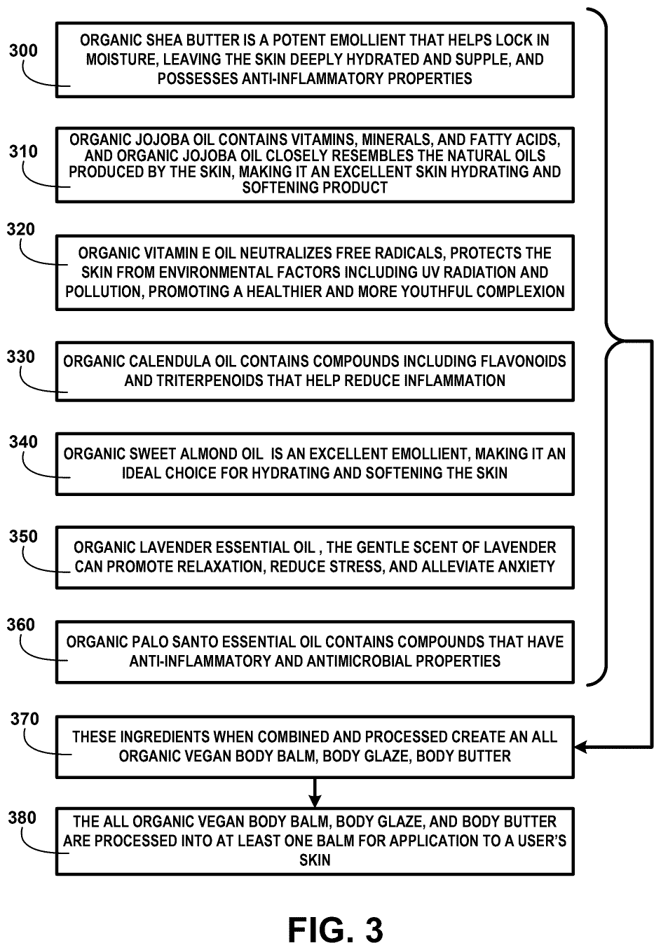

FIG. 3 shows a block diagram of an overview of the ingredient characteristics of one embodiment. FIG. 3 shows ingredient characteristics that describe each ingredient's contribution to moisturizing skin.

In one embodiment, organic shea butter 110 is used as an ingredient of the all-organic vegan body balm, body glaze, body butter, and body skin topical cosmetic balm formulation 100. The all-organic vegan body balm, body glaze, body butter, and body skin topical cosmetic balm formulation 100 of FIG. 1 with organic shea butter 110 can be used as a potent emollient that helps lock in moisture, leaving the skin deeply hydrated and supple. Also, the all-organic vegan body balm, body glaze, body butter, and body skin topical cosmetic balm formulation 100 of FIG. 1 with organic shea butter 110 can obtain anti-inflammatory properties 300. all-organic vegan body balm, body glaze, body butter, and body skin topical cosmetic balm formulation 100 of FIG. 1 can have shea butter extracted from the nuts of the African shea tree, which is a rich and nourishing natural ingredient that will allow a multitude of benefits for skin and hair. The all-organic vegan body balm, body glaze, body butter, and body skin topical cosmetic balm formulation 100 of FIG. 1 with organic shea butter 110 will allow exceptional moisturizing properties. Since organic shea butter 110 is abundant in fatty acids and vitamins, the all-organic vegan body balm, body glaze, body butter, and body skin topical cosmetic balm formulation 100 of FIG. 1 can be used as a potent emollient to help lock in moisture, leaving the skin deeply hydrated and supple. The all-organic vegan body balm, body glaze, body butter, and body skin topical cosmetic balm formulation 100 of FIG. 1 with organic shea butter 110 can be used regularly for dry or sensitive skin, to help provide a protective barrier against harsh environmental elements.

In addition, the all-organic vegan body balm, body glaze, body butter, and body skin topical cosmetic balm formulation 100 of FIG. 1 with organic shea butter 110 can possess anti-inflammatory properties to make it an excellent choice for soothing irritated or inflamed skin. The all-organic vegan body balm, body glaze, body butter, and body skin topical cosmetic balm formulation 100 of FIG. 1 with organic shea butter 110 can be used to help with eczema, dermatitis, or sunburn to promote relief and skin healing. The natural compounds in organic shea butter 110, including cinnamic acid and lupeol, contribute to its anti-inflammatory effects. Since organic shea butter 110 is renowned for its ability to promote skin elasticity and collagen production, the presence of vitamins A and E in organic shea butter 110 supports the skin's natural renewal process, aiding in the maintenance of skin complexion.

The all-organic vegan body balm, body glaze, body butter, and body skin topical cosmetic balm formulation 100 of FIG. 1 with organic shea butter 110 is a versatile product that can be used in various ways. For example, the all-organic vegan body balm, body glaze, body butter, and body skin topical cosmetic balm formulation 100 of FIG. 1 can be used as body butter and applied directly to the skin, providing hydration and a natural glow. The all-organic vegan body balm, body glaze, body butter, and body skin topical cosmetic balm formulation 100 of FIG. 1 can also be used as a base for DIY skincare products, such as lip balms. In hair care, the all-organic vegan body balm, body glaze, body butter, and body skin topical cosmetic balm formulation 100 of FIG. 1 with organic shea butter 110 can be applied to dry or damaged hair to restore moisture and enhance shine.

The all-organic vegan body balm, body glaze, body butter, and body skin topical cosmetic balm formulation 100 of FIG. 1 with organic shea butter 110 can be warmed between the palms before application. It can be gently massaged into the skin or hair until fully absorbed. The all-organic vegan body balm, body glaze, body butter, and body skin topical cosmetic balm formulation 100 of FIG. 1 with organic shea butter 110 can be applied daily as part of a skincare or hair care routine, and its versatility allows it to be customized for specific needs or concerns.

Thus, all-organic vegan body balm, body glaze, body butter, and body skin topical cosmetic balm formulation 100 of FIG. 1 with organic shea butter 110 as one example ingredient in the present invention can be used as an ingredient that is a natural and effective solution for promoting skin and hair health. From its intense moisturizing and anti-inflammatory properties to its ability to support skin elasticity, organic shea butter 110 offers a holistic approach to skincare and hair care for the all-organic vegan body balm, body glaze, body butter, and body skin topical cosmetic balm formulation 100 of FIG. 1. Whether used on its own or as part of a DIY beauty routine, the all-organic vegan body balm, body glaze, body butter, and body skin topical cosmetic balm formulation 100 of FIG. 1 provides a nourishing and enriching experience to enhance skin and hair.

In one embodiment, organic jojoba oil 120 is used as an ingredient in the all-organic vegan body balm, body glaze, body butter, and body skin topical cosmetic balm formulation 100. Organic jojoba oil 120 can contain vitamins, minerals, and fatty acids, and organic jojoba oil 120 closely resembles the natural oils produced by the skin, making it an excellent skin hydrating and softening product 310. In the present invention, the organic jojoba oil 120 can be derived from the seeds of the jojoba plant (Simmondsia chinensis) and is a versatile and popular natural ingredient that can offer numerous benefits for skincare and hair care. Rich in vitamins, minerals, and fatty acids, organic jojoba oil 120 closely resembles the natural oils produced by our skin, making it an excellent skin-hydrating and softening product. One of its key benefits is its ability to regulate sebum production, making it suitable for all skin types, including oily and acne-prone skin. This non-comedogenic organic jojoba oil 120 absorbs easily without clogging pores, leaving the skin nourished and hydrated.

Beyond its moisturizing properties, organic jojoba oil 120 can be used in the present invention as an anti-inflammatory and antioxidant ingredient. Organic jojoba oil 120 contains natural compounds that soothe irritated skin, making it an effective remedy for conditions such as eczema and psoriasis. Additionally, the antioxidants in organic jojoba oil 120 of the present invention can help protect the skin from environmental stressors, reducing the signs of aging and promoting a healthy complexion.

In the realm of hair care, the organic jojoba oil 120 of the present invention can be used as a conditioning treatment ingredient. The all-organic vegan body balm, body glaze, body butter, and body skin topical cosmetic balm formulation 100 of FIG. 1 with organic jojoba oil 120 can be applied to the hair and scalp to moisturize, strengthen, and add shine. The organic jojoba oil 120 has a molecular structure closely resembling the natural sebum produced by the scalp to allow it to help dry or damage hair. The all-organic vegan body balm, body glaze, body butter, and body skin topical cosmetic balm formulation 100 of FIG. 1 with organic jojoba oil 120 can also help balance oil production in the scalp, addressing issues such as dandruff and promote overall hair health.

The all-organic vegan body balm, body glaze, body butter, and body skin topical cosmetic balm formulation 100 of FIG. 1 with organic jojoba oil 120 can be used by applying as massage onto clean, damp skin as a daily skin hydrating and softening product or added to existing skincare products for an extra boost. As a hair treatment, the all-organic vegan body balm, body glaze, body butter, and body skin topical cosmetic balm formulation 100 of FIG. 1 can be applied in a small amount to the ends of damp hair or used as an overnight mask for deep conditioning treatment. The all-organic vegan body balm, body glaze, body butter, and body skin topical cosmetic balm formulation 100 of FIG. 1 can be extended to its use as a carrier oil for essential oils or as a base for natural DIY beauty recipes. By incorporating organic jojoba oil 120 into the all-organic vegan body balm, body glaze, body butter, and body skin topical cosmetic balm formulation 100 of FIG. 1, a natural and sustainable approach can be obtained.

In one embodiment, organic vitamin E oil 130 is used as one ingredient in the all-organic vegan body balm, body glaze, body butter, and body skin topical cosmetic balm formulation 100 of FIG. 1. The all-organic vegan body balm, body glaze, body butter, and body skin topical cosmetic balm formulation 100 of FIG. 1 with organic vitamin E oil 130 neutralizes free radicals and protects the skin from environmental factors including UV radiation and pollution, promoting a healthier and more youthful complexion 320. Organic vitamin E oil can be used for its antioxidant properties, as a skincare ingredient with numerous benefits for skin health. One of the primary advantages of organic vitamin E oil 130 is its ability to neutralize free radicals, which are molecules that can damage skin cells and contribute to signs of aging. By combating oxidative stress, organic vitamin E oil 130 helps protect the skin from environmental factors such as UV radiation and pollution, promoting a healthier and more youthful complexion.

In addition to its antioxidant properties, organic vitamin E oil 130 contributes to its moisturizing effects. The all-organic vegan body balm, body glaze, body butter, and body skin topical cosmetic balm formulation 100 of FIG. 1 with organic vitamin E oil 130 is highly emollient, meaning it helps lock in moisture and prevents dehydration for individuals with dry or flaky skin. Regular application of organic vitamin E oil 130 can contribute to smoother, softer skin, reducing the appearance of fine lines and wrinkles.

Organic vitamin E oil 130 also plays a role in promoting skin healing. It has anti-inflammatory properties that can soothe irritated or damaged skin. When applied topically, organic vitamin E oil 130 may aid in reducing redness, and inflammation, and promoting the healing of wounds or scars. This makes it a popular choice for addressing minor skin issues, such as cuts, scrapes, or sunburns.

Furthermore, organic vitamin E oil 130 has been found to support collagen production, a key protein responsible for maintaining skin elasticity and firmness. By encouraging collagen synthesis, organic vitamin E contributes to the prevention of sagging skin and helps maintain a more youthful appearance. This makes it a valuable addition to anti-aging skincare routines.

The all-organic vegan body balm, body glaze, body butter, and body skin topical cosmetic balm formulation 100 of FIG. 1 with organic vitamin E oil 130 is suitable for various skin types, and its non-comedogenic nature means it is unlikely to clog pores. It can be applied topically in the all-organic vegan body balm, body glaze, body butter, and body skin topical cosmetic balm formulation 100 of FIG. 1 directly to the skin. Many individuals also use organic vitamin E oil 130 to address specific skin concerns, applying it to areas prone to dryness, wrinkles, or scars.

The all-organic vegan body balm, body glaze, body butter, and body skin topical cosmetic balm formulation 100 of FIG. 1 with organic vitamin E oil 130 can be used effectively, a small amount can be applied to the face or body and gently massaged into the skin. It can be used as part of a nightly skincare routine or applied as needed for targeted treatment. The all-organic vegan body balm, body glaze, body butter, and body skin topical cosmetic balm formulation 100 of FIG. 1 is mixed with other carrier oils or serums to create a customized skincare blend.

Organic vitamin E oil 130 is a versatile and beneficial ingredient in skincare. From its antioxidant and anti-inflammatory properties to its moisturizing and collagen-boosting effects, organic vitamin E oil 130 offers a holistic approach to promoting skin health. Whether used as a standalone treatment or incorporated into a comprehensive skincare regimen, vitamin E oil provides a natural and effective solution for maintaining and enhancing the vitality of the skin.

In one embodiment, organic calendula oil 140 is used as an ingredient for the all-organic vegan body balm, body glaze, body butter, and body skin topical cosmetic balm formulation 100 of FIG. 1. The all-organic vegan body balm, body glaze, body butter, and body skin topical cosmetic balm formulation 100 of FIG. 1 with organic calendula oil 140 contains compounds including flavonoids and triterpenoids that help reduce inflammation 330. Organic calendula oil 140, derived from the bright orange and yellow flowers of the organic Calendula officinalis plant, provides benefits for skin health and overall well-being. One of its primary advantages lies in its anti-inflammatory properties. Organic calendula oil 140 contains compounds such as flavonoids and triterpenoids that help reduce inflammation, beneficial in soothing irritated skin. It is often used to alleviate conditions like eczema, dermatitis, and minor skin irritations.

Additionally, the all-organic vegan body balm, body glaze, body butter, and body skin topical cosmetic balm formulation 100 of FIG. 1 with organic calendula oil 140 is used for its wound-healing properties. Its ability to stimulate collagen production aids in the repair and regeneration of damaged skin tissue. Applying organic calendula oil 140 to minor cuts, scrapes, or bruises may accelerate the healing process, minimizing scarring and promoting healthier-looking skin. The all-organic vegan body balm, body glaze, body butter, and body skin topical cosmetic balm formulation 100 of FIG. 1 with the organic calendula oil's antimicrobial properties further contribute to its efficacy in preventing infections in minor wounds.

The all-organic vegan body balm, body glaze, body butter, and body skin topical cosmetic balm formulation 100 of FIG. 1 with organic calendula oil 140 is a potent skin hydrating and softening ingredient, making it beneficial for dry or chapped skin. The emollient nature of the organic calendula oil 140 helps lock in moisture, keeping the skin hydrated and supple. Regular application of the all-organic vegan body balm, body glaze, body butter, and body skin topical cosmetic balm formulation 100 of FIG. 1 with organic calendula oil 140 may contribute to softer, smoother skin, making it a valuable addition to skincare routines, especially in harsh weather conditions that can contribute to skin dryness.

For skin conditions such as acne, the all-organic vegan body balm, body glaze, body butter, and body skin topical cosmetic balm formulation 100 of FIG. 1 with organic calendula oil 140 can be used to provide anti-inflammatory and antibacterial properties that are beneficial to those conditions. The organic calendula oil 140 helps soothe inflamed skin and may aid in reducing acne-related redness. Using the all-organic vegan body balm, body glaze, body butter, and body skin topical cosmetic balm formulation 100 of FIG. 1 with organic calendula oil 140 in conjunction with a regular skincare routine can contribute to a clearer complexion and healthier-looking skin.

The all-organic vegan body balm, body glaze, body butter, and body skin topical cosmetic balm formulation 100 of FIG. 1 with organic calendula oil 140 can be used topically in various ways to harness its benefits. It can be applied directly to the skin as a skin hydrating and softening treatment with healing properties. The all-organic vegan body balm, body glaze, body butter, and body skin topical cosmetic balm formulation 100 of FIG. 1 with organic calendula oil 140 can also be used in massage oils, particularly for individuals with sensitive or irritated skin. When selecting organic calendula oil 140, it's essential to choose a high-quality, cold-pressed version to ensure the preservation of its beneficial compounds.

The all-organic vegan body balm, body glaze, body butter, and body skin topical cosmetic balm formulation 100 of FIG. 1 with organic calendula oil 140 can be used as a natural remedy with a multitude of benefits for skin health. Its anti-inflammatory, wound-healing, and moisturizing properties make it a valuable addition to skincare routines. Whether used for soothing irritated skin, promoting wound healing, or enhancing overall skin health, the all-organic vegan body balm, body glaze, body butter, and body skin topical cosmetic balm formulation 100 of FIG. 1 with organic calendula oil 140 offers a gentle and effective solution for a variety of skincare needs.

In one embodiment, organic sweet almond oil 150 is used as an ingredient for the all-organic vegan body balm, body glaze, body butter, and body skin topical cosmetic balm formulation 100 of FIG. 1. The all-organic vegan body balm, body glaze, body butter, and body skin topical cosmetic balm formulation 100 of FIG. 1 with organic sweet almond oil 150 is an emollient, making it an ideal choice for hydrating and softening the skin 340. Organic sweet almond oil 150 can be used that's extracted from the kernels of almond trees. It is a versatile and nourishing oil with a wide range of benefits for skin, hair, and overall health. One of its key advantages lies in its rich moisturizing properties. Organic sweet almond oil 150 is an emollient so it can be used for hydrating and softening the skin. Regular application of the all-organic vegan body balm, body glaze, body butter, and body skin topical cosmetic balm formulation 100 of FIG. 1 with organic sweet almond oil 150 can help prevent dryness, leaving the skin feeling supple and smooth.

In addition to its moisturizing qualities, the all-organic vegan body balm, body glaze, body butter, and body skin topical cosmetic balm formulation 100 of FIG. 1 with organic sweet almond oil 150 can be used to promote skin health. Since it contains vitamins A and E, as well as fatty acids, it will have antioxidant and anti-inflammatory properties. These components work together to protect the skin from environmental stressors, reduce inflammation, and support the skin's natural repair process. As a result, the all-organic vegan body balm, body glaze, body butter, and body skin topical cosmetic balm formulation 100 of FIG. 1 with organic sweet almond oil 150 is often used to soothe irritated or sensitive skin and may aid in managing conditions such as eczema or dermatitis.

The all-organic vegan body balm, body glaze, body butter, and body skin topical cosmetic balm formulation 100 of FIG. 1 with organic sweet almond oil 150 is also beneficial for hair care. When applied to the hair and scalp, it can help nourish and strengthen strands, making hair appear shinier and healthier. Massaging a small amount of the all-organic vegan body balm, body glaze, body butter, and body skin topical cosmetic balm formulation 100 of FIG. 1 with organic sweet almond oil 150 into the scalp may also contribute to improved blood circulation, promoting healthy hair growth and reducing dandruff. The all-organic vegan body balm, body glaze, body butter, and body skin topical cosmetic balm formulation 100 of FIG. 1 with organic sweet almond oil 150 lightweight and non-greasy nature make it a popular choice for leave-in conditioners or hair masks.

The all-organic vegan body balm, body glaze, body butter, and body skin topical cosmetic balm formulation 100 of FIG. 1 with organic sweet almond oil 150 can be applied directly to the skin as a skin hydrating and softening ingredient with a customized skincare blend. It can also be used as a carrier oil for essential oils in aromatherapy. In hair care, all-organic vegan body balm, body glaze, body butter, and body skin topical cosmetic balm formulation 100 of FIG. 1 with organic sweet almond oil 150 can be applied as a leave-in treatment or incorporated into homemade hair masks. Additionally, the all-organic vegan body balm, body glaze, body butter, and body skin topical cosmetic balm formulation 100 of FIG. 1 with organic sweet almond oil 150 can be added to bathwater for a moisturizing and relaxing bathing experience.

The all-organic vegan body balm, body glaze, body butter, and body skin topical cosmetic balm formulation 100 of FIG. 1 with organic sweet almond oil 150 is a natural and versatile remedy with a range of benefits for skin and hair. Its moisturizing, antioxidant, and anti-inflammatory properties make it a valuable addition to daily beauty and self-care routines. The all-organic vegan body balm, body glaze, body butter, and body skin topical cosmetic balm formulation 100 of FIG. 1 with organic sweet almond oil 150 is a customized blend, of sweet almond oil that offers a nourishing and gentle solution for promoting overall health and well-being.

In one embodiment, organic lavender essential oil 160 is used as an ingredient for the all-organic vegan body balm, body glaze, body butter, and body skin topical cosmetic balm formulation 100 of FIG. 1. The all-organic vegan body balm, body glaze, body butter, and body skin topical cosmetic balm formulation 100 of FIG. 1 with organic lavender essential oil 160 has a scent of lavender and can promote relaxation, reduce stress, and alleviate anxiety 350. The all-organic vegan body balm, body glaze, body butter, and body skin topical cosmetic balm formulation 100 of FIG. 1 with the organic lavender essential oil 160 can be derived from the lavender plant. It can be used in aromatherapy and holistic health practices. One of its primary advantages lies in its calming and soothing properties. Inhaling the gentle scent of lavender can promote relaxation, reduce stress, and alleviate anxiety.

Additionally, organic lavender essential oil 160 possesses notable anti-inflammatory and analgesic properties. When all-organic vegan body balm, body glaze, body butter, and body skin topical cosmetic balm formulation 100 of FIG. 1 with organic lavender essential oil 160 is applied topically, it may assist in relieving minor skin irritations, insect bites, or muscle discomfort. The organic lavender essential oil 160 also has antimicrobial attributes further making it a natural option for promoting skin health, aiding in the healing of minor wounds, and preventing infections.

Furthermore, the all-organic vegan body balm, body glaze, body butter, and body skin topical cosmetic balm formulation 100 of FIG. 1 with organic lavender essential oil 160 can be used to promote restful sleep. The calming scent can be diffused in the bedroom to create a serene environment conducive to a good night's sleep.

Organic lavender essential oil 160 also has natural insect-repelling properties. As such, the all-organic vegan body balm, body glaze, body butter, and body skin topical cosmetic balm formulation 100 of FIG. 1 with organic lavender essential oil 160 can be an eco-friendly natural insect-repellant. The all-organic vegan body balm, body glaze, body butter, and body skin topical cosmetic balm formulation 100 of FIG. 1 with organic lavender essential oil 160 can be used to promote relaxation and sleep and to provide relief for minor skin issues. The all-organic vegan body balm, body glaze, body butter, and body skin topical cosmetic balm formulation 100 of FIG. 1 with organic lavender essential oil 160 can be used as aromatherapy skincare.

Organic Palo Santo essential oil 170 can be used as an ingredient for the all-organic vegan body balm, body glaze, body butter, and body skin topical cosmetic balm formulation 100 of FIG. 1. The all-organic vegan body balm, body glaze, body butter, and body skin topical cosmetic balm formulation 100 of FIG. 1 with organic Palo Santo essential oil 170 contains compounds that have anti-inflammatory and antimicrobial properties 360. Organic Palo Santo essential oil 170, has spiritual and therapeutic benefits. The name “Palo Santo” translates to “holy wood” in Spanish, and its usage dates back centuries in traditional rituals and ceremonies. One of the primary benefits of Palo Santo wood is its ability to cleanse and purify the energy in a space. The fragrance of Palo Santo included as an ingredient of the all-organic vegan body balm, body glaze, body butter, and body skin topical cosmetic balm formulation 100 can be used as a spiritual tool for meditation, prayer, or simply to create a sacred atmosphere.

Beyond its spiritual properties, Palo Santo wood is also known for its natural aromatic qualities. The wood emits a sweet and uplifting fragrance when burned, creating a pleasant and calming environment. This aromatic profile makes Palo Santo wood a popular choice for those seeking a natural alternative to traditional incense. The scent is believed to have mood-enhancing effects, promoting relaxation and reducing stress and anxiety.

In addition to its spiritual and aromatic properties, the Palo Santo ingredient can be used in the all-organic vegan body balm, body glaze, body butter, and body skin topical cosmetic balm formulation 100 of FIG. 1 as an anti-inflammatory and antimicrobial. The all-organic vegan body balm, body glaze, body butter, and body skin topical cosmetic balm formulation 100 of FIG. 1 with Palo Santo essential oil 170 can be used for topical applications, such as soothing minor skin irritations or relieving muscle tension through massage. Integrating the all-organic vegan body balm, body glaze, body butter, and body skin topical cosmetic balm formulation 100 of FIG. 1 with organic Palo Santo essential oil 170 into a skincare routine can provide benefits for skin care treatments.

These ingredients are combined in the all-organic vegan body balm, body glaze, body butter, and body skin topical cosmetic balm formulation 100 of FIG. 1 and processed to create an all-organic vegan body balm, body glaze, and body butter 370. The all-organic vegan body balm, body glaze, and body butter ingredients are processed for application to a user's skin 380.

### Combining Organic Ingredients:

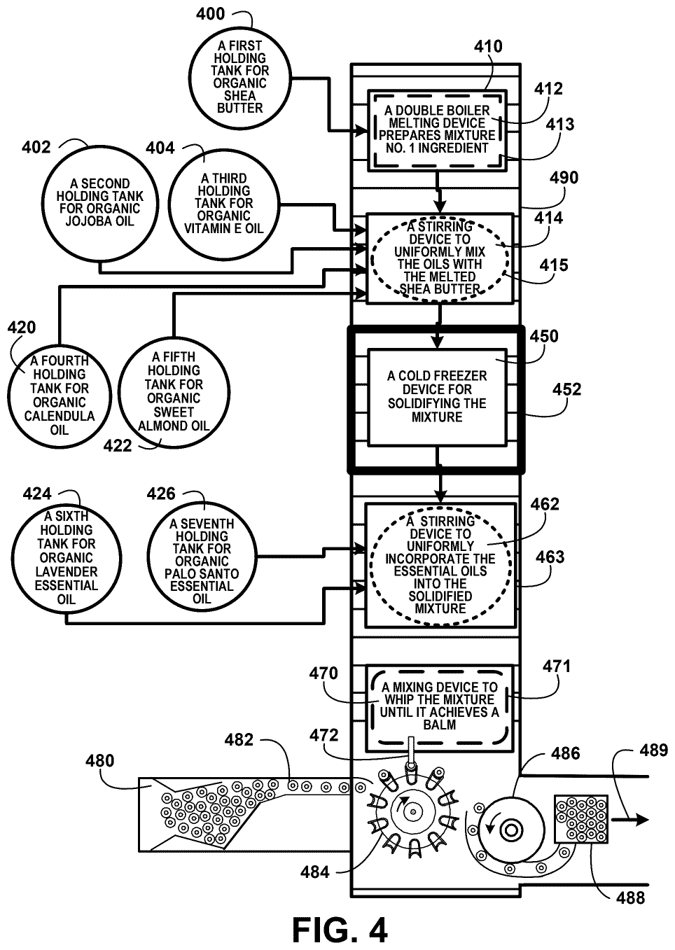

FIG. 4 shows for illustrative purposes only an example of combining organic ingredients of one embodiment. FIG. 4 shows the first holding tank for organic shea butter 400. A predetermined measured amount of shea butter is placed into a mixing vat 410. A double boiler melting device 413 is used to melt the shea butter into the mixing vat 410 as a liquid. The double boiler melting device prepares mixture no. 1 ingredient 412. A conveyor belt 490 moves the mixing vat 410 along the processing line. Predetermined measured amounts of the ingredients are dispensed from the holding tanks into the mixing vat 410 during the preparation processes.

A second holding tank for organic jojoba oil 402, a third holding tank for organic vitamin E oil 404, a fourth holding tank for organic calendula oil 420, and a fifth holding tank for organic sweet almond oil 422 dispense the predetermined measured amounts of the oils. A first stirring device to uniformly mix the oils with the melted shea butter 414. The first stirring device 415 automatically is lowered into the mixing vat 410 when in position for stirring and retracts after a predetermined stirring time.

A cold freezer device for solidifying the mixture 450 is sized to allow the mixing vat of the stirred ingredients to be conveyed into the refrigeration device 452 for cooling the mixture.

A sixth holding tank for organic lavender essential oil 424 and a seventh holding tank for organic Palo Santo essential oil 426 are dispensed in predetermined measured amounts of the essential oils into the mixing vat 410. A second stirring device to uniformly incorporate the essential oils into the solidified mixture 462 is automatically lowered into the mixing vat 410 when the vat is in position. The second stirring device 463 is retracted after a predetermined stirring time.

A mixing device to whip the mixture until it achieves a body balm 470 is automatically lowered into the mixing vat 410 when the vat is in position. The mixing device 471 is retracted when the predetermined whipping time is complete. A dispensing device 472 is automatically lowered into the mixing vat 410 when the vat is in position.

A container alignment device 480 moves a plurality of the containers 482 for proper placement in a container filling device 484. The container filling device 484 rotates each container beneath the dispensing device 472. The dispensing device 472 pumps a predetermined measured amount of the whipped balm into the container.

A lid-securing device (not shown) secures a lid to the container and seals the lid. The lid for capping the container depends on the type of container being filled. In various embodiments, the container may be a dark glass jar and the lid a metal or plastic screw-on cap. In another embodiment, the container may be a plastic container where a user can remove the lid and use a finger to wipe the balm, then spread the balm over the affected skin area. Next in the process, the container is moved to a container labeling device 486 to affix a label to the container, wherein the label includes regulatory standards applicable to show the ingredients and other required information.

A container boxing device 488 is filled with the container in predetermined numbers and when full the box is sealed, and the box is moved along the conveyor to a shipping area 489 of one embodiment. Combining organic ingredients like shea butter, jojoba oil, vitamin E oil, calendula oil, sweet almond oil, lavender essential oil, and Palo Santo essential oil results in a beneficial skincare product. The combining of the ingredients is a step-by-step process to mix the organic ingredients into, for example, a clear or translucent balm that can be applied to the body, such as the skin.

In one embodiment, the holding tanks are used to collect and gather the organic shea butter, organic jojoba oil, organic vitamin E oil, organic calendula oil, organic sweet almond oil, organic lavender essential oil, and organic Palo Santo essential oil. All ingredients are of high quality and sourced organically for the best skincare benefits. The quantities of each ingredient are measured to meet a basic recipe and are adjusted to meet a production batch quantity for a specific number of containers to be produced. Each holding tank is equipped with a dispenser with a predetermined quantity dispensing capability to match the batch size. The double boiler volume is sized to contain various batch sizes. The temperature to melt the Shea Butter is adjusted to match the batch quantity to prevent overheating the Shea Butter. The double boiler is further equipped to include a stirring device to occasionally the Shea Butter until it becomes a liquid. The liquid oils are then incorporated together, including the jojoba oil, vitamin E oil, calendula oil, and sweet almond oil into the mixture and it is uniformly blended into the liquid Shea Butter by stirring well as the liquid oils are added to the mixture.

The mixture is cooled for a brief period to reduce the temperature after the shea butter has been melted. This further is performed to preserve the therapeutic properties of the essential oils. Incorporation of the lavender essential oil and Palo Santo essential oil into the cooled mixture can be made with predetermined numbers of drops or equivalent volumes. The number of drops may be adjusted to adjust fragrance scent intensity. The mixture is whipped using the mixing device is used to whip the mixture until it achieves a balm consistency to produce a smooth and luxurious texture. The whipping time may be varied to achieve different consistencies. The product container can be used to protect the balm from light and preserve the quality of the ingredients.

In one embodiment, all of these ingredients are combined are discussed above to create the all-organic vegan body balm, body glaze, body butter, and body skin topical cosmetic balm formulation 100 of FIG. 1 as an organic balm with the moisturizing properties of shea butter, the nourishing effects of various carrier oils, and the aromatic benefits of essential oils. The all-organic vegan body balm, body glaze, body butter, and body skin topical cosmetic balm formulation 100 offers a customized and natural skincare solution, free from synthetic additives and tailored to a user's specific preferences.

### Ingredients by Weight:

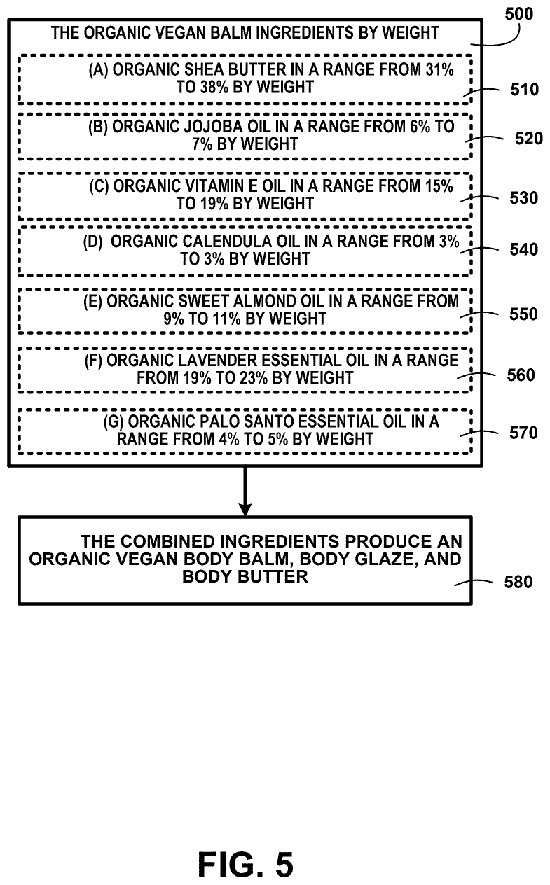

FIG. 5 shows a block diagram of an overview of ingredients by weight of one embodiment. FIG. 5 shows the organic vegan balm ingredients by weight 500. In one embodiment, the formulation for the all-organic vegan body balm, body glaze, body butter, and body skin topical cosmetic balm formulation 100 of FIG. 1 can be an organic vegan skin hydrating and softening product that can have numerous different ranges of weight for the ingredients.

The ranges are met for various production batch quantities. In one embodiment, the ingredient weight proportions are as follows: (a) organic shea butter in a range from 31% to 38% by weight 510, (b) organic jojoba oil in a range from 6% to 7% by weight 520, (c) organic vitamin E oil in a range from 15% to 19% by weight 530, (d) organic calendula oil in a range from 3% to 3% by weight 540, (e) organic sweet almond oil in a range from 9% to 11% by weight 550, (f) organic lavender essential oil in a range from 19% to 23% by weight 560, and (g) organic Palo Santo essential oil in a range from 4% to 5% by weight 570.

The ingredients and their proportions in FIG. 5 are a working example and are for illustrative purposes only. The combined organic vegan skin hydrating and softening ingredients in FIG. 5 can produce the all-organic vegan body balm, body glaze, body butter, and body skin topical cosmetic balm formulation 100 of FIG. 1 as an organic vegan body balm, body glaze, and body butter 580 of one embodiment.

### User Sharing Application Results on Social Media:

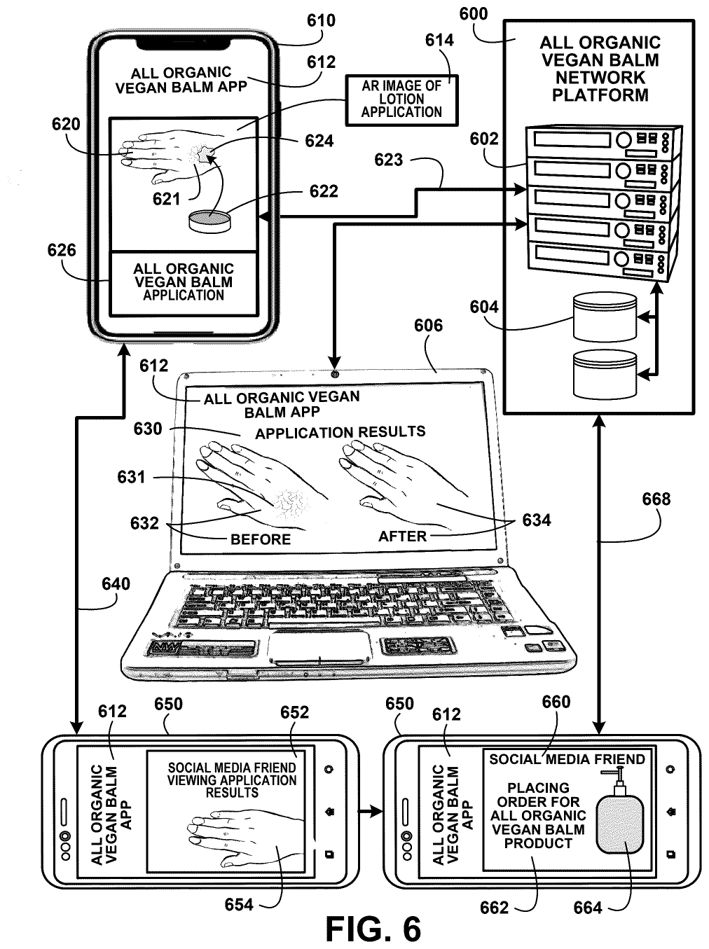

FIG. 6 shows for illustrative purposes only an example of a user sharing application results on social media of one embodiment. FIG. 6 shows a platform 600 having a plurality of servers 602, a plurality of databases 604, and at least one computer 606. A user mobile device having augmented reality (AR) 610 and an app 612 are wirelessly coupled to the platform 600. In one embodiment, the user captures an AR image of a balm application 614 showing a user's hand in an AR virtual environment 620 and an AR virtual balm container and application of the balm upon a hand 622.

The captured AR image displays an AR virtual all-organic vegan balm being applied 624 to the user's hand. The user is capturing the AR images to track the progress of the all-organic vegan balm's effectiveness in hydrating and softening the dry skin on the user's hand. The all-organic vegan balm application 626 is being applied to the affected skin. The AR image of a balm application captured images is wirelessly transmitted 623 to the plurality of servers 602 and recorded in the plurality of databases 604. The AR images of the balm application captured images are shown in a time-lapse progress on a network computer 606.

The all-organic vegan balm app 612 is shown displaying the application results in 630 on the user hand dry skin 631 in a user's hand before all organic vegan balm application 632 and the tracked progress in images of the user's hand after all organic vegan balm applications 634 being sent by the user wirelessly communicating with a social media friend 640. The platform 600 can include in one embodiment an artificial intelligence and machine learning system that tracks, monitors and learns certain aspects of the user's results based on human interaction of self-reported information and learned tracked and monitored information from the AR images and other gathered data to make live and human interactive recommendations on how and when to use the balm for best results.

The user social media friend mobile device 650 having the all-organic vegan balm app 612 is displayed to the social media friend viewing application results 652. The images of the user's hand after all organic vegan balm applications 634 show the user's hand showing dry skin cleared up 654. The user's social media friend mobile device 650 shows the social media friend 660 placing an order for all organic vegan balm product 662 of the AR image of an all-organic vegan balm product 664. The user's social media friend's mobile device placed the order wirelessly transmitted to the all-organic vegan balm network platform 668 where the social media friend 660 can make the payment for the order and receive a message on a delivery date for the order being placed of one embodiment.

### all Organic Vegan Balm Product Types:

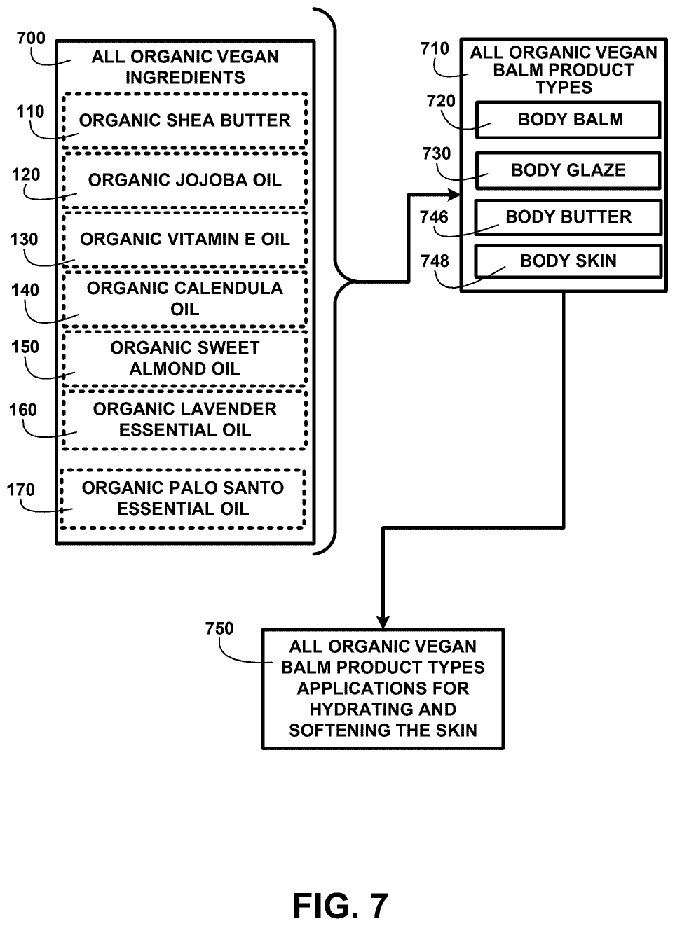

FIG. 7 shows a block diagram of an overview of the all-organic vegan balm product types of one embodiment. FIG. 7 shows a listing of the all-organic vegan ingredients 700. The all-organic vegan ingredients 700 include organic shea butter 110, organic jojoba oil 120, organic vitamin E oil 130, organic calendula oil 140, organic sweet almond oil 150, organic lavender essential oil 160, and organic Palo Santo essential oil 170.

The all-organic vegan ingredients 700 are blended in predetermined quantities to create various all-organic vegan balm product types 710. The various all-organic vegan balm product types 710 include body balm 720, body glaze 730, body butter 746, and body skin 748. The all-organic vegan balm product type 710 is packaged into various types of containers including pump containers, squeeze tubes, jars, spray bottles, and others suitable for the product consistency. The purpose of the all-organic vegan balm product types of applications for hydrating and softening the skin 750 is to provide relief to users of one embodiment.

The foregoing has described the principles, embodiments, and modes of operation of the present invention. However, the invention should not be construed as being limited to the particular embodiments discussed. The above-described embodiments should be regarded as illustrative rather than restrictive, and it should be appreciated that variations may be made in those embodiments by workers skilled in the art without departing from the scope of the present invention as defined by the following claims.

What is claimed is:

1. A balm composition of ingredients, comprising:
formulating a composition of ingredients that comprise,
(a) shea butter in a range from 31% to 38% by weight;
(b) jojoba oil;
(c) vitamin E oil;
(d) calendula oil;
(e) sweet almond oil;
(f) lavender essential oil; and
(g) palo santo essential oil.

2. The balm composition of ingredients of claim 1, wherein the sweet almond oil ingredient is in a range from 9% to 11% by weight.

3. The balm composition of ingredients of claim 1, wherein the lavender essential oil is in a range from 19% to 23% by weight.

4. The balm composition of ingredients of claim 1, wherein the palo santo essential oil is in a range from 4% to 5% by weight.

5. The balm composition of ingredients of claim 1, wherein the calendula oil is in a range from 3% to 3% by weight.

6. The balm composition of ingredients of claim 1, wherein the vitamin E oil is in a range from 15% to 19% by weight.

7. The balm composition of ingredients of claim 1, wherein the jojoba oil is in a range from 6% to 7% by weight.

8. The balm composition of ingredients of claim 1, wherein the shea butter, jojoba oil, vitamin E oil, calendula oil, sweet almond oil, lavender essential oil, and palo santo essential oil are organic and vegan ingredients.

9. The balm composition of ingredients of claim 1, further comprising utilizing a mobile device with an augmented reality application operating on the mobile device to capture augmented reality images of a user to track progress and use of the body balm and further comprising an artificial intelligence and machine learning system coupled to the mobile device that tracks, monitors and learns certain aspects of a user's results based on human interaction of self-reported information from the user and learned tracked and monitored information from the augmented reality application to make human interactive recommendations on how and when to use the balm composition for best results.

10. A balm composition of ingredients, comprising:
shea butter in a range from 31% to 38% by weight;
jojoba oil;
vitamin E oil;
calendula oil;
sweet almond oil in a range from 9% to 11% by weight;
lavender essential oil in a range from 19% to 23% by weight; and
palo santo essential oil.

11. The balm composition of ingredients of claim 10, wherein the shea butter, jojoba oil, vitamin E oil, calendula oil, sweet almond oil, lavender essential oil, and palo santo essential oil are organic and vegan ingredients.

12. The balm composition of ingredients of claim 10, wherein the palo santo essential oil is in a range from 4% to 5% by weight.

13. The balm composition of ingredients of claim 10, wherein the calendula oil is in a range from 3% to 3% by weight.

14. The balm composition of ingredients of claim 10, wherein the vitamin E oil is in a range from 15% to 19% by weight.

15. The balm composition of ingredients of claim 1, wherein the jojoba oil is in a range from 6% to 7% by weight.

16. The balm composition of ingredients of claim 1, further comprising a mobile device with an augmented reality application operating on the mobile device configured to capture augmented reality images of a user to track progress and use of the body balm and further comprising an artificial intelligence and machine learning system coupled to the mobile device that tracks, monitors and learns certain aspects of a user's results based on human interaction of self-reported information from the user and learned tracked and monitored information from the augmented reality application to make human interactive recommendations on how and when to use the balm composition for best results.

17. A balm composition of ingredients, comprising:
jojoba oil;
shea butter in a range from 31% to 38% by weight;
vitamin E oil in a range from 15% to 19% by weight;
calendula oil in a range from 3% to 3% by weight;
sweet almond oil in a range from 9% to 11% by weight;
lavender essential oil in a range from 19% to 23% by weight; and
palo santo essential oil in a range from 4% to 5% by weight.

18. The balm composition of ingredients of claim 17, wherein the shea butter, jojoba oil, vitamin E oil, calendula oil, sweet almond oil, lavender essential oil, and palo santo essential oil are organic and vegan ingredients.

19. The balm composition of ingredients of claim 17, wherein the jojoba oil is in a range from 6% to 7% by weight.

20. The balm composition of ingredients of claim 17, further comprising a mobile device with an augmented reality application operating on the mobile device configured to capture augmented reality images of a user to track progress and use of the body balm and further comprising an artificial intelligence and machine learning system coupled to the mobile device that tracks, monitors and learns certain aspects of a user's results based on human interaction of self-reported information from the user and learned tracked and monitored information from the augmented reality application to make human interactive recommendations on how and when to use the balm composition for best results.
